(19) United States
(12) Patent Application Publication
(10) Pub. No.: US 2025/0391540 A1
(43) Pub. Date: Dec. 25, 2025

(54) SYSTEM AND METHOD FOR ENHANCEMENT OF COPING SKILLS

(71) Applicant: Gopikrishna Yeleswarapu, Birmingham, AL (US)

(72) Inventor: Gopikrishna Yeleswarapu, Birmingham, AL (US)

(21) Appl. No.: 19/247,166

(22) Filed: Jun. 24, 2025

Related U.S. Application Data
(60) Provisional application No. 63/663,453, filed on Jun. 24, 2024.

(51) Int. Cl. G16H 20/70; G16H 10/60

(52) U.S. Cl. CPC G16H 20/70; G16H 10/60

(57) ABSTRACT

The present disclosure pertains to a system and method for creating social stories to enhance coping skills, particularly for neurodivergent individuals. The system includes a profile builder to generate user profiles based on user information, a designer module to create characters and scenarios tailored to the user profile, and a story engine that utilizes Artificial Intelligence models, such as Large Language Models (LLMs), to generate social stories. A privacy module ensures compliance with privacy regulations by anonymizing Personally Identifiable Information (PII) and Personal Health Information (PHI) during story generation and reintegrating the anonymized data afterward for personalization. The method involves building user profiles, designing actors and scenarios, anonymizing data, generating social stories using AI, and optionally refining them through human review. This approach automates the creation of social stories, reducing costs and wait times, while maintaining data security and personalization, thereby addressing the needs of neurodivergent individuals.

### CROSS-REFERENCE TO RELATED APPLICATIONS

This application claims benefit to Provisional Application No. 63/663,453, filed Jun. 24, 2024, the contents of which are herein incorporated by reference.

### BACKGROUND OF THE INVENTION

The present invention relates to therapeutic systems and methods, and more particularly, to a therapeutic system and method for enhancing coping skills in neurodivergent person(s).

Neurodivergent describes a person whose brain develops or works differently. Neurodivergent disorders include dyslexia, autism spectrum disorder (ASD), attention deficit hyperactivity disorder (ADHD), obsessive compulsive disorder (OCD), Tourette's Syndrome, and more. Neurodivergent persons have different strengths and struggles from persons whose brains develop or work more typically. Traditionally, neurodivergent person(s) may have information processing variances, enhanced perceptual abilities, and/or intense focusing and attention abilities. Additionally, neurodivergent person(s) may have sensor sensitivities, trouble with emotional regulation, and may utilize internal coping mechanisms such as repetitive, or ritualistic behavior.

A social story is a mechanism utilized by professionals to aid neurodivergent person(s), typically those with autism, to understand and deal with complex life situations. Social stories are important because they can help neurodivergent person(s) relate to situations, surroundings, and people, aiding them in learning what to do, and what not to do in unfamiliar situations. Traditionally, social stories are written by trained professionals, as a poorly written social story can damage a person's self-esteem or put them in danger. However, the lack of trained professionals in conjunction with an increased awareness, and subsequent identification, of neurodivergent person(s) can lead to long wait times for the creation of social stories, and high costs associated with their creation. As can be seen, there is a need for an improved system and method for creating social stories.

### SUMMARY OF THE INVENTION

In one embodiment, the disclosure includes a system for creating one or more social stories. The system comprises a profile builder configured to receive user information and build a corresponding user profile, a designer module configured to generate a character or a scenario based on the user profile, a story engine configured to generate the social stories using the user profile, the generated character, or the generated scenario, and a privacy module configured to remove personally identifiable information from the user profile, character, or scenario prior to generating the social stories and to reintegrate such information after the generation. At least one of these modules is stored in memory and executed by a processor.

In another embodiment, the disclosure includes a computer-implemented method for creating one or more social stories. The method comprises creating at least one user profile based on user information, generating an actor or one or more scenarios from the user profile, and creating one or more requests that include the actor or the scenario. The method further includes anonymizing the requests to remove personally identifiable information or personal health information to form filtered requests, generating one or more anonymized social stories based on the filtered requests, and de-anonymizing the anonymized social stories to yield the final social stories. In some embodiments, the step of generating the anonymized social stories comprises submitting the filtered requests to artificial intelligence models, such as large language models, and generating the anonymized social stories using these models prior to outputting the final social stories.

### BRIEF DESCRIPTION OF THE DRAWINGS

FIG. 1 is a block diagram of an embodiment of a system for social story creation, according to aspects of the present invention; and

FIG. 2 is a flow diagram of an embodiment of a method for social story creation, according to aspects of the present invention.

### DETAILED DESCRIPTION OF THE INVENTION

The following detailed description is of the best currently contemplated modes of carrying out exemplary embodiments of the disclosure. The description is not to be taken in a limiting sense but is made merely for the purpose of illustrating the general principles of the disclosure, since the scope of the disclosure is best defined by the appended claims.

Social stories are expensive and time consuming to create often requiring a professional drafter. Currently, there are no automated systems for creating social stories because no systems offer compliance with regulatory requirements such as the Health Information Portability Act, or Federal Education Rights and Privacy Act.

Broadly, an embodiment of the present disclosure provides a system and method for creation of social stories while maintaining information privacy and security.

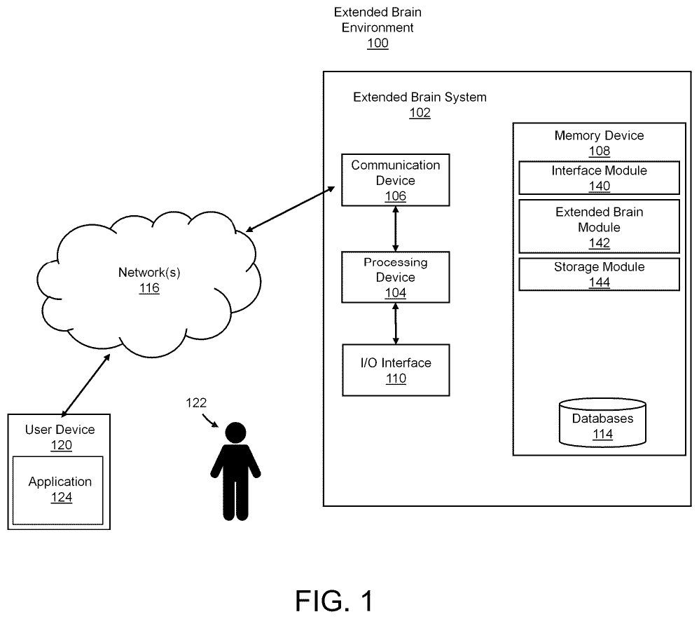

Referring now to FIG. 1, FIG. 1 illustrates an embodiment of a system for social story creation, hereinafter ExtendedBrain, according to aspects of the present disclosure. The present invention provides for the automated creation of social stories while maintaining information privacy and security according to regulatory requirements. While FIG. 1 illustrates various components of the ExtendedBrain System, additional components can be added, and existing components can be removed.

As illustrated in FIG. 1, the ExtendedBrain system 102 includes one or more processing devices, herein processing device 104, coupled to a communication device 106. The processing device 104 is also coupled to a memory device 108, and an input/output (“I/O”) interface 110. In embodiments, the communication device 106 enables ExtendedBrain system 102 to communicate with other devices and systems via one or more networks 116. The ExtendedBrain system 102 can communicate with a user device 120 via the network 116. A user 122 can utilize the user device 120 to communicate with the ExtendedBrain system 102. The user device 120 can include one or more electronic devices such as a laptop computer, a desktop computer, a tablet computer, a smartphone, a thin client, a smart appliance, and the like. While FIG. 1 illustrates one user device 120, the ExtendedBrain environment 100 can include multiple user devices operated by the user 122 or operated by other users.

According to the aspects of the present disclosure, the ExtendedBrain system 102 enables the user 122, operating a copy of an application 124 executing on the user device 120, to communicate with the ExtendedBrain system 102 and leverage the service provided by the ExtendedBrain system 102. The ExtendedBrain system 102 is configured to create social stories by seamlessly integrating user and actor profiles with advanced data management techniques. At its core, the ExtendedBrain system 102 is configured to provide intermediary services between user 122 and AI-based large language models (LLMs) such as ChatGPT or Google Gemini. The ExtendedBrain system 102 is configured to effectively harness personal and sensitive information while safeguarding privacy through de-identification of personally identifiable information (PII) and protected health information (PHI).

To perform the process described herein, the ExtendedBrain system 102 can store and execute an Interface module 140, a ExtendedBrain module 142, and an Storage module 144 to perform the processes and methods described herein. The Interface module 140, the ExtendedBrain module 142, and the Storage module 144 can be stored in the memory device 108. The Interface module 140, the ExtendedBrain module 142, and the Storage module 144 can include the necessary logic, instructions, and/or programming to perform the processes and methods described in further detail below. The Interface module 140, the ExtendedBrain module 142, and the Storage module 144 can be written in any programming language.

In embodiments, the application 124 can be a specifically designed application that operates with the ExtendedBrain system 102 to perform the processes and methods described herein. In embodiments, the application 124 can be a third-party application, such as a web browser, that communicates with the ExtendedBrain system 102 to perform the processes and methods described herein. The memory device 108 can also include one or more databases 114 that store information and data associated with the process and methods described below in further detail.

According to aspects of the present disclosure, the ExtendedBrain system 102, for example, via the Interface module 140, provides unique interfaces that allow the user 122 to create/update/develop social stories, build user profiles, design actors and scenarios, generate actors and scenarios, integrate actor and user profiles, access or otherwise utilize Large Language Models, access databases 114, review or otherwise edit social stories, etc. The Interface module 140 operates to generate and provide graphical user interfaces (GUIs) to the application 122, for example, menus, widgets, text, images, fields, etc., as described below in further detail. The GUIs generated by the Interface module 140 can be interactive. The ExtendedBrain system 102, for example, via the Interface module 140, also provide one or more application programming interface (APIs) that provide connection points for one or more application, e.g., the application 124.

In embodiments, the Interface module 140 can implement voice control aspects into the interfaces provided. For example, the user can navigate the interfaces of the ExtendedBrain system 102 using the audio input device of the user device 120. The interface module 140 can implement one or more chat-bots to deliver conversational input and output to a user.

According to aspects of the present disclosure, the ExtendedBrain system 102, for example, via the ExtendedBrain Module 142, provides unique processing modules and interfaces that allow the user 122 to create/update/develop social stories, build user profiles, design actors and scenarios, generate actors and scenarios, integrate actor and user profiles, access or otherwise utilize Large Language Models, access databases 114, review or otherwise edit social stories, etc. The ExtendedBrain module 142 can include a plurality of sub-modules configured to carry out tasks necessary for the operation of the present invention.

A first sub-module of the ExtendedBrain system 102 can be a profile builder sub-module configured to allow user 122 to build at least one user profile. In embodiments, the at least one user profile can include demographic data, user preferences, user interests and other personal information necessary to create a customized social story. Advantageously, creation of a user profile ensures that any story created based on the user profile aligns with the user's preferences and backgrounds.

A second sub-module of the ExtendedBrain system 102 can be a designer sub-module configured to design and generate at least one actor and/or at least one scenario. In embodiments, the at least one actor can be built or designed based on preferences of the user indicated in the user profile. Additionally, the at least one scenario can be created and can be structured around a social interaction, or situation, relevant to the user's profile. Advantageously, creation of actors and/or scenarios in this manner ensures relevance and engagement for a user interacting with a story.

A third sub-module of the ExtendedBrain system 102 can be a story engine interface sub-module. In embodiments, the story engine interface sub-module gathers information from the first sub-module and the second sub-module and provides the gathered information to a Large Language Model (LLM) to generate a social story. In embodiments, the LLM can be a pre-built LLM such as ChatGPT, or Google's Gemini, and can receive gathered information and create a social story based on the gathered information.

A fourth sub-module of the ExtendedBrain system 102 can be a data privacy sub-module. In embodiments, the data privacy sub-module can filter, or otherwise redact, personally identifiable information from user profile, and/or the gathered information before the information is provided to the LLM by the story interface sub-module. Additionally, the data privacy sub-module can re-add, or reintegrate, personally identifiable information to the social story returned by the LLM. In embodiments, the data privacy sub-module can be split into data deidentification and data reidentification. In embodiments, data deidentification removes personal information and sends the deidentified data to the LLM or other AI system to generate, or enhance, a social story thereby leveraging structured input while ensuring anonymity. Once a social story is received, data reidentification re-enters PII ensuring the social story can be linked back to the correct user profile and can provide personalized content. Advantageously, the data privacy sub-module can ensure personal data is protected during processing, while enabling personalization of the file social story by reintroducing personal information.

In addition to the above sub-modules, a number of optional sub-modules can be included in the present invention. For example, a Human review sub-module can be provided, which is configured to allow a user to modify, or otherwise, edit a social story generated by the LLM, or other AI. Advantageously, the human review sub-module can enhance the story's quality by incorporating human insight into the AI-generated narratives. Additionally, a feedback loop sub-module can be provided, which is configured to refine social stories based on user provided feedback. Advantageously, the user feedback loop sub-module can improve user satisfaction and engagement with social stories.

According to aspects of the present disclosure, the ExtendedBrain system 102, for example, via the Storage module 144 can provide necessary storage and retrieval of data across different stages of social story creation, ensuring data integrity and continuity.

The processing device 104, the communication device 106, the memory device 108, and the I/O interface 110 can be interconnected via a system bus. The system bus can be and/or include a control bus, a data bus, and address bus, and so forth. The processing device 104 can be and/or include a processor, a microprocessor, a computer processing unit

(“CPU”), a graphics processing unit (“GPU”), a neural processing unit, a physics processing unit, a digital signal processor, an image signal processor, a synergistic processing element, a field-programmable gate array (“FPGA”), a sound chip, a multi-core processor, and so forth. As used herein, “processor,” “processing component,” “processing device,” and/or “processing unit” can be used generically to refer to any or all of the aforementioned specific devices, elements, and/or features of the processing device. While FIG. 1 illustrates a single processing device 104, the ExtendedBrain system 102 can include multiple processing devices 104, whether the same type or different types.

The memory device 108 can be and/or include computerized storage medium capable of storing electronic data temporarily, semi-permanently, or permanently. The memory device 108 can be or include a computer processing unit register, a cache memory, a magnetic disk, an optical disk, a solid-state drive, and so forth. The memory device can be and/or include random access memory (“RAM”), read-only memory (“ROM”), static RAM, dynamic RAM, masked ROM, programmable ROM, erasable and programmable ROM, electrically erasable and programmable ROM, and so forth. As used herein, “memory,” “memory component,” “memory device,” and/or “memory unit” can be used generically to refer to any or all of the aforementioned specific devices, elements, and/or features of the memory device. While FIG. 1 illustrates a single memory device 108, the ExtendedBrain system 102 can include multiple memory devices 108, whether the same type or different types.

The communication device 106 enables the ExtendedBrain system 102 to communicate with other devices and systems. The communication device 104 can include, for example, a networking chip, one or more antennas, and/or one or more communication ports. The communication device 106 can generate radio frequency (RF) signals and transmit the RF signals via one or more of the antennas. The communication device 106 can generate electronic signals and transmit the RF signals via one or more of the communication ports. The communication device 106 can receive the RF signals from one or more of the communication ports. The electronic signals can be transmitted to and/or from a communication hardline by the communication ports. The communication device 106 can generate optical signals and transmit the optical signals to one or more of the communication ports. The communication device 106 can receive the optical signals and/or can generate one or more digital signals based on the optical signals. The optical signals can be transmitted to and/or received from a communication hardline by the communication port, and/or the optical signals can be transmitted and/or received across open space by the communication device 106.

The communication device 106 can include hardware and/or software for generating and communicating signals over a direct and/or indirect network communication link. As used herein, a direct link can include a link between two devices where information is communicated from one device to the other without passing through an intermediary. For example, the direct link can include a BluetoothTM connection, a Zigbee connection, a Wifi DirectTM connection, a near-field communications (“NFC”) connection, an infrared connection, a wired universal serial bus (“USB”) connection, an ethernet cable connection, a fiber-optic connection, a firewire connection, a microwire connection, and so forth. In another example, the direct link can include a cable on a bus network. An indirect link can include a link between two or more devices where data can pass through an intermediary, such as a router, before being received by an intended recipient of the data. For example, the indirect link can include a WiFi connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (“GSM”) standard, a code division multiple access (“CDMA”) standard such as the universal mobile telecommunications standard, an orthogonal frequency division multiple access (“OFDMA”) standard such as the long term evolution (“LTE”) standard, and so forth.

The ExtendedBrain system 102 can communicate with one or more network resources via the network 116. The one or more network resources can include external databases, social media platforms, search engines, file servers, web servers, APIs, SDKs or any type of computerized resource that can communicate with the ExtendedBrain system 102 via the network 116.

As described above, the ExtendedBrain system 102 can include hardware components to perform the processes described herein. In embodiments, one or more of components, hardware, and/or functionality of the ExtendedBrain system 102 can be hosted and/or instantiated on a “cloud” or “cloud service.” As used herein, a “cloud” or “cloud service” can include a collection of computer resources that can be invoked to instantiate a virtual machine, application instance, process, data storage, serverless compute or other resources for a limited or defined duration. The collection of resources supporting a cloud can include a set of computer hardware and software configured to deliver computing components needed to instantiate a virtual machine, application instance, process, data storage, or other resources. For example, one group of computer hardware and software can host and serve an operating system or components thereof to deliver to and instantiate a virtual machine. Another group of computer hardware and software can accept requests to host computing cycles or processor time, to supply a defined level of processing power for a virtual machine. A further group of computer hardware and software can host and serve applications to load on an instantiation of a virtual machine, such as an email client, a browser application, a messaging application, or other applications or software. Other types of computer hardware and software are possible.

In embodiments, the components and functionality of the ExtendedBrain system 102 can be and/or include a “server” device. The term server can refer to functionality of a device and/or an application operating on a device. The server device can include a physical server, a virtual server, and/or cloud server. For example, the server device can include one or more bare-metal servers such as single-tenant servers or multiple-tenant servers. In another example, the server device can include a bare metal server partitioned into two or more virtual servers. The virtual servers can include separate operating systems and/or applications from each other. In yet another example, the server device can include a virtual server distributed on a cluster of networked physical servers. The virtual servers can include an operating system and/or one or more applications installed on the virtual server and distributed across the cluster of networked physical servers. In yet another example, the server device can include more than one virtual server distributed across a cluster of networked physical servers.

Various aspects of the systems described herein can be referred to as “information,” “content,” and/or “data.” Content and/or data can be used to refer generically to modes of storing and/or conveying information. Accordingly, data can refer to textual entries in a table of a database. Content and/or data can refer to alphanumeric characters stored in a database. Content and/or data can refer to machine-readable code. Content and/or data can refer to images. Content and/or data can refer to audio and/or video. Content and/or data can refer to, more broadly, a sequence of one or more symbols. The symbols can be binary. Content and/or data can refer to a machine state that is computer-readable. Content and/or data can refer to human-readable text.

Various of the devices in the ExtendedBrain environment 100, including the ExtendedBrain system 102 and/or the user device 120 can provide I/O devices for outputting information in a format perceptible by a user and receiving input from the user. For example, the ExtendedBrain system 102 can communicate with the I/O devices via the I/O interface 110. The I/O devices can display graphical user interfaces (“GUIs”) generated by the ExtendedBrain system 102. The I/O devices can include a display screen such as a light-emitting diode (“LED”) display, an organic LED (“OLED”) display, an active-matrix OLED (“AMOLED”) display, a liquid crystal display (“LCD”), a thin-film transistor (“TFT”) LCD, a plasma display, a quantum dot (“QLED”) display, and so forth. The I/O devices can include an acoustic element such as a speaker, a microphone, and so forth. The I/O devices can include a button, a switch, a keyboard, a touch-sensitive surface, a touchscreen, a camera, a fingerprint scanner, and so forth. The touchscreen can include a resistive touchscreen, a capacitive touchscreen, and so forth.

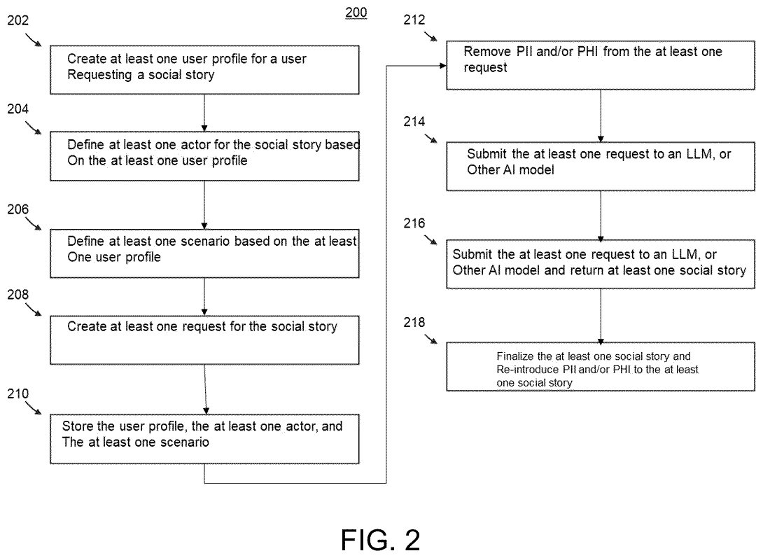

FIG. 2 illustrates a method 200 for creating a social story, according to aspects of the present disclosure. While FIG. 2 illustrates various stages of the method 200, additional stages can be added, and existing stages can be removed and/or reordered.

Method 200 can begin at step 202 where a user profile is built. In embodiments, a user can enter information such as demographics, interest, preferences, etc., to create a user profile. In embodiments, the created user profile provides inputs to the system necessary for creating a social story, such as defining actors, and scenarios specific to the user.

Once the user profile is built, one or more foundational story elements are created based on the user profile. At step 204 a first foundational story element is created as at least one actor. In embodiments, the at least one actor for a social story can be defined based on information provided in the user profile. In embodiments, the at least one actor is defined based on information from the user profile such that the at least one actor aligns with the preferences of the user.

In an exemplary embodiment, actor information in the user profile includes at least three data points, as actor information. First, actor identifying information is provided, which includes demographic information such as place of living, grade in school. Second, one or more physically identifying characteristics of the actor are provided such as race, hair color, eye color, etc. In embodiments, the physically identifying information is provided to one or more Generative AI system which provide personalized images based on the information. Third a psychological profile of the actor is developed to gather behavioral traits, triggers, calming strategies and special interests. Once provided, the actor information is converted into a description paragraph and stored in storage devices such as a database to be used at the time when a story is generated.

At step 206 a second foundational story element is created as at least one scenario. In embodiments, the at least one or more scenario can be defined. In embodiments, the at least one scenario can be an outline of a plot and setting of a requested social story. Once the one or more foundational story elements are created, one or more social stories are generated based on one or more of the foundational story elements.

At step 208, a request can be issued to create a social story. In embodiments, the request can include the one or more foundational story elements, such as, the at least one actor, the at least one user profile, and the at least one scenario. Additionally, the request can form an outline to be submitted to an LLM, or other AI, as a prompt to create a social story consistent with the request.

At step 208, when a request is issued to create a social story, the system initiates a complex integration process that begins with retrieving and incorporating previously created user profile data, including demographic information such as age, gender, and developmental stage, alongside psychological profile characteristics encompassing anxiety levels, social skills, communication preferences, individual triggers, comprehension level, and language capabilities. Simultaneously, the system accesses the generated actor profile that serves as the visual representation of the user, incorporating physical characteristics that mirror or relate to the target individual, behavioral traits consistent with the user profile, visual styling appropriate for the user's age and preferences, and contextual appearance settings for the specific scenario.

The system then processes the selected scenario template and customizes it based on the specific topic or situation chosen from available categories such as dental visits, peer interactions, or transitions, while incorporating environmental factors relevant to the user's circumstances, behavioral objectives and learning goals, and potential anxiety triggers or challenges specific to the scenario. These foundational elements are subsequently combined through a sophisticated prompt engineering process to create a structured request for the Large Language Model (LLM), which includes formatted user profile data as context parameters, actor profile specifications for consistent character representation, scenario parameters and environmental details, narrative structure requirements and evidence-based formatting guidelines, and specific behavioral objectives and therapeutic goals. The system formulates comprehensive requests for both textual and visual content generation, incorporating text generation parameters for age-appropriate language and structure, image generation specifications for illustrations that match the actor profile, consistency requirements across multiple scenes or pages, and therapeutic effectiveness criteria based on social narrative research.

At step 210, the system implements a robust data storage and persistence framework that maintains comprehensive user profile data including demographic and psychological profile information, user preferences and customization settings, historical usage patterns and effectiveness metrics, all while ensuring privacy-compliant data handling in accordance with HIPAA and FERPA requirements. The actor profile data is persistently stored comprising visual character specifications and rendering parameters, behavioral characteristics and personality traits, consistency markers for cross-narrative recognition, and version control for profile updates and modifications. Scenario information is archived including template definitions and customization parameters, specific scenario instances created for individual users, effectiveness tracking data and outcome measurements, and categorization and indexing for future retrieval. The system maintains a comprehensive repository of created social stories that includes complete narrative text with formatting and structure, associated visual content and illustrations, generation parameters and source foundational elements, and usage tracking and effectiveness metrics.

The technical implementation establishes and maintains complex relational data mapping between stored elements, linking user profiles to their associated actor profiles, connecting scenarios to appropriate user demographics and needs, mapping generated stories to their foundational components, and creating comprehensive audit trails for compliance and quality assurance. The database architecture utilizes a structured approach where user profiles are stored with encrypted personally identifiable information, actor profiles maintain visual and behavioral consistency parameters, scenarios are templated for reusability while allowing customization, and generated content is linked to source elements for complete traceability. The AI integration process follows a systematic approach incorporating structured prompt formatting to ensure consistent input parameters, multi-turn conversations for iterative refinement when necessary, quality validation before content delivery, and fallback mechanisms to handle edge cases and generation failures. Throughout this entire process, the privacy and compliance framework ensures data handling adheres to regulatory requirements through encryption of sensitive user information, access controls and audit logging, data retention policies aligned with healthcare regulations, and secure transmission protocols for all data exchanges, thereby ensuring that social story generation is both therapeutically effective and technically robust while providing personalized interventions and maintaining data security and regulatory compliance.

At step 212, the system implements a comprehensive privacy enhancement framework that provides sophisticated data filtering, obfuscation, and selective removal processes to ensure user privacy while maintaining therapeutic effectiveness of the generated social narratives. The system employs advanced algorithms specifically designed to identify Protected Health Information (PHI) and Personally Identifiable Information (PII) within the foundational story elements, utilizing pattern recognition and contextual analysis to detect names, addresses, specific locations, medical information, and other sensitive identifiers that could compromise user privacy or violate regulatory standards. The PHI identification process leverages algorithmic recognition systems that systematically scan through user profiles, actor profiles, and scenario data to identify names and places with high accuracy, employing natural language processing techniques, named entity recognition, and contextual semantic analysis to distinguish between generic references and specific personal identifiers.

Upon identification of sensitive information, the system applies a sophisticated combination of anonymization technologies that appropriately adjust names and other identifiable information through a proprietary replacement algorithm, while maintaining contextual integrity and narrative coherence essential for therapeutic effectiveness. The anonymization process operates through intelligent substitution mechanisms that replace identified names with demographically appropriate alternatives, substitute specific locations with generic or fictitious equivalents when the narrative's therapeutic value is not dependent on precise geographical information, and preserve location-specific information only when the social story's effectiveness is fundamentally tied to particular places, such as scenarios involving familiar environments or specific institutional settings. The system's proprietary algorithm combines the actor profile characteristics, social story prompt parameters, and scenario specifications with ExtendedBrain's meta-model to create a comprehensive anonymization schema that effectively obscures user identity while preserving all elements necessary for personalized therapeutic intervention.

The anonymization process systematically replaces names and other identifiable information with contextually appropriate substitute identifiers, ensuring that demographic consistency, age-appropriate naming conventions, cultural sensitivity, and relational accuracy are maintained throughout the transformation. During the anonymization period, all original identifying information is securely stored in a structured data mapping system maintained in Random Access Memory (RAM) on a processing server, utilizing temporary storage protocols that ensure data persistence only for the duration of the generation process while implementing robust security measures to prevent unauthorized access or data persistence beyond the required processing timeframe. This structured data map maintains precise correlations between original identifiers and their anonymized counterparts, enabling accurate reidentification upon completion of the content generation process while ensuring that sensitive information never persists in permanent storage systems accessible to external AI models.

At step 214, once the comprehensive filtering and anonymization process is completed, the sanitized request containing all necessary therapeutic and contextual information but devoid of identifying personal data is submitted to the Large Language Model or other artificial intelligence systems as a structured prompt designed to generate at least one personalized social story. The filtered prompt maintains all essential elements required for effective narrative generation, including anonymized character specifications, scenario parameters, behavioral objectives, therapeutic goals, and contextual environmental factors, while ensuring complete compliance with regulatory standards including HIPAA and FERPA requirements. The LLM processes this privacy-compliant prompt to create comprehensive social narratives that incorporate age-appropriate language, behaviorally relevant scenarios, therapeutically sound interventions, and engaging visual descriptions, all while operating on completely anonymized data that cannot be traced back to the original user.

At step 218, the system initiates the finalization and reidentification process, systematically restoring the original personally identifiable information to create a fully customized social story tailored specifically for the target user. The reidentification process accesses the structured data map maintained in RAM memory to retrieve the original names, locations, and other identifying information, then systematically replaces the anonymized placeholders with the authentic personal details to create a narrative that resonates with the user's actual environment, relationships, and experiences. This reidentification ensures that the social story maintains maximum therapeutic impact by incorporating familiar names, recognizable locations, and personally relevant details while having been generated through a completely privacy-compliant process that never exposed sensitive information to external AI systems. Upon completion of the reidentification process, the structured data map is immediately purged from memory, ensuring that no temporary storage of sensitive information persists beyond the generation cycle, thereby maintaining the highest standards of data privacy and regulatory compliance throughout the entire social story creation workflow. At step 216, following the reidentification process, the system provides an optional human refinement capability that allows qualified professionals to review, modify, and enhance the fully personalized social story to address potential oversights, correct factual errors, improve therapeutic effectiveness, or incorporate specialized clinical insights that automated systems might overlook. This human intervention process operates on the reidentified content, with authorized human reviewers having access to the fully personalized social story containing the original personally identifiable information, enabling them to provide valuable clinical expertise, therapeutic guidance, narrative enhancement, and quality assurance while working with the complete contextual information necessary to optimize the social story's effectiveness for the intended therapeutic outcomes. The human refinement process may include adjustments to language complexity, behavioral intervention strategies, scenario accuracy, visual description enhancement, and therapeutic goal alignment, all performed with full access to the personalized content.

As used in the description herein and throughout the claims that follow, “a”, “an”, and “the” include plural references unless the context clearly dictates otherwise. Also, as used in the description herein and throughout the claims that follow, the meaning of “in” includes “in” and “on” unless the context clearly dictates otherwise. While the above is a complete description of specific examples of the disclosure, additional examples are also possible. Thus, the above description should not be taken as limiting the scope of the disclosure which is defined by the appended claims along with their full scope of equivalents.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements. As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

It should be understood, of course, that the foregoing relates to exemplary embodiments of the disclosure and that modifications can be made without departing from the spirit and scope of the disclosure as set forth in the following claims.

What is claimed is:

1. A system for creating one or more social stories, comprising:
a profile builder configured to receive at least one user information and build a user profile based on the at least one user information;
a designer module configured to generate one or more of:
at least one character, or at least one scenario, based on the user profile;
a story engine configured to generate the one or more social stories based on one or more of:
the user profile, the at least one character, or the at least one scenario; and
a privacy module configured to, prior to generating the one or more social stories, remove personally identifiable information from the user profile, the at least one character, and the at least one scenario, and to reintegrate, after generating the one or more social stories, personally identifiable information removed from the user profile, the at least one character, and the at least one scenario, wherein at least one of the profile builder, the designer module, the story engine, or the privacy module are stored in memory and executed by a processor.

2. A computer implemented method executed by a processor for creating one or more social stories, comprising:
creating, by the processing, at least one user profile based on at least one user information;
creating, based on the at least one user profile, one or more of: an actor, or at least one scenario;
creating one or more requests, wherein the one or more requests include one or more of: the actor, or the at least one scenario;
anonymizing the one or more requests to remove one or more of Personally Identifiable Information (PII), or Personal Health Information (PHI) to form one or more filtered requests;
creating one or more anonymized social stories based on the one or more filtered requests;
de-anonymizing the one or more anonymized social stories to form the one or more social stories; and
outputting the one or more social stories.

3. The method of claim 2, wherein creating the one or more anonymized social stories, further comprises:
submitting the one or more filtered requests to one or more Artificial Intelligence Models; and
generating, using the one or more Artificial Intelligence Models, the one or more anonymized social stories.

4. The method of claim 3, wherein the one or more Artificial Intelligence Models are Large Language Models.
